(19) United States
(12) Patent Application Publication
(10) Pub. No.: US 2026/0114831 A1
(43) Pub. Date: Apr. 30, 2026

(54) COMPUTER PROGRAM, INFORMATION PROCESSING APPARATUS, AND INFORMATION PROCESSING METHOD

(71) Applicant: TERUMO KABUSHIKI KAISHA, Tokyo (JP)

(72) Inventors: Tomomi SEGAMI, Odawara-city (JP); Yuya KANAZAWA, Fuji-city (JP)

(73) Assignee: TERUMO KABUSHIKI KAISHA, Tokyo (JP)

(21) Appl. No.: 19/003,311

(22) Filed: Dec. 27, 2024

Related U.S. Application Data
(63) Continuation of application No. PCT/JP2023/015634, filed on Apr. 19, 2023.

(30) Foreign Application Priority Data
Aug. 31, 2022 (JP) 2022-138469

(51) Int. Cl. A61B 6/00; A61B 6/12; A61B 6/46; A61B 6/50; A61M 25/01; A61M 25/09; G16H 20/40

(52) U.S. Cl. CPC A61B 6/5217; A61B 6/12; A61B 6/463; A61B 6/487; A61B 6/504; G16H 20/40; A61M 2025/0166; A61M 25/09

(57) ABSTRACT

A non-transitory computer-readable medium, an information processing apparatus, and an information processing method for assisting selection of a device to be used for intravascular treatment. The non-transitory computer-readable medium storing a computer program that causes a computer to execute processing including acquiring lesion information indicating a state of a lesion portion included in a blood vessel of a patient and bias information of a wire to be inserted into the blood vessel and specifying a recommended device recommended to be used for treatment on the lesion portion, based on the lesion information and the bias information, and device information of a treatment device to be used for intravascular treatment.

### CROSS-REFERENCES TO RELATED APPLICATIONS

This application is a continuation of International Application No. PCT/JP2023/015634 filed on Apr. 19, 2023, which claims priority to Japanese Application No. 2022-138469 filed on Aug. 31, 2022, the entire content of both of which is incorporated herein by reference.

### TECHNOLOGICAL FIELD

The present disclosure generally relates to a non-transitory computer-readable medium storing a computer program, an information processing apparatus, and an information processing method related to intravascular treatment.

### BACKGROUND DISCUSSION

As one of methods for treating a lesion portion such as coarctation or occlusion occurred in a blood vessel, there is intravascular treatment for performing treatment from an inside of the blood vessel using a device percutaneously inserted into the blood vessel. In the intravascular treatment, a guide wire is inserted into the blood vessel, a device such as an atherectomy device is moved to the lesion portion along the guide wire, and treatment such as excising of the lesion portion is performed. In the intravascular treatment, an image in which a blood vessel is fluoroscopically viewed from outside using X rays and an image in a blood vessel in which an inside of the blood vessel is imaged by a method such as optical frequency domain imaging (OFDI) or intravascular ultrasound (IVUS) are used. Japanese Patent Application Publication No. 2021-104337 A discloses an example of a technology for performing the intravascular treatment.

In intravascular treatment, it is necessary to use an appropriate device according to a state of a lesion portion. For example, in a case where treatment for excising an eccentric lesion portion is performed, it is desirable to use a device that can excise the eccentric lesion portion and does not affect a portion other than the lesion portion. It may be not possible to verify what treatment effect can be obtained when the device is used unless the device is actually used, selection of the appropriate device is not easy.

### SUMMARY

A non-transitory computer-readable medium storing a computer program, an information processing apparatus, and an information processing method for assisting selection of a device used for intravascular treatment are disclosed.

(1) A non-transitory computer-readable medium storing a computer program according to an embodiment of the present disclosure causes a computer to execute processing including acquiring lesion information indicating a state of a lesion portion included in a blood vessel of a patient and bias information of a wire to be inserted into the blood vessel, and specifying a recommended device recommended to be used for treatment on the lesion portion, based on the lesion information and the bias information, and device information of a treatment device to be used for intravascular treatment.

(2) The non-transitory computer-readable medium according to (1), causes the computer to execute processing further including displaying a blood vessel cross-sectional image representing a cross section of the blood vessel and superimposing and displaying a direction and a strength of pressing the wire against an inner surface of the blood vessel at a position where the blood vessel cross-sectional image is obtained, included in the bias information, on the blood vessel cross-sectional image.

(3) The non-transitory computer-readable medium according to (1) or (2), causes the computer to execute processing further including displaying the blood vessel cross-sectional image representing the cross section of the blood vessel, displaying a fluoroscopic image in which the blood vessel is fluoroscopically viewed from outside of the patient, and superimposing and displaying a trajectory of a plurality of positions in the blood vessel where the blood vessel cross-sectional image is created and a position where the displayed blood vessel cross-sectional image is created, on the fluoroscopic image.

(4) The non-transitory computer-readable medium according to (3) superimposes and displays a position of the lesion portion on the fluoroscopic image.

(5) The non-transitory computer-readable medium according to any one of (1) to (4), displays a type and a severity of the lesion portion included in the lesion information.

(6) The non-transitory computer-readable medium according to any one of (1) to (5), causes the computer to execute processing including displaying the specified recommended device.

(7) The non-transitory computer-readable medium according to any one of (1) to (6), causes the computer to execute processing further including displaying the blood vessel cross-sectional image representing the cross section of the blood vessel and superimposing and displaying a treatment range by the specified recommended device on the blood vessel cross-sectional image.

(8) The non-transitory computer-readable medium according to any one of (1) to (7), causes the computer to execute processing further including displaying the blood vessel cross-sectional image representing the cross section of the blood vessel, receiving designation of a use device to be used for treatment on the lesion portion, and superimposing and displaying a treatable range of the lesion portion by the use device on the blood vessel cross-sectional image.

(9) The non-transitory computer-readable medium according to (8), causes the computer to execute processing further including displaying a fluoroscopic image in which the blood vessel is fluoroscopically viewed from outside of the patient and superimposing and displaying a position of the use device in the blood vessel when the use device is used, on the fluoroscopic image.

(10) The non-transitory computer-readable medium according to any one of (1) to (9), causes the computer to execute processing further including making a display indicating that a wire bias is inappropriate, in a case where the bias information is not suitable for the intravascular treatment.

(11) The non-transitory computer-readable medium according to (10), causes the computer to execute processing further including displaying a proposal of processing for enabling intravascular treatment, including a change in a route through which the wire passes in the blood vessel and a change in the wire.

(12) The non-transitory computer-readable medium according to any one of (1) to (11), causes the computer to execute processing further including acquiring the bias information, by generating the bias information including a direction and a strength of pressing the wire against the inner surface of the blood vessel at each position of the blood vessel, based on data representing a three-dimensional shape of the blood vessel created based on the fluoroscopic image, data representing a three-dimensional shape of the wire in a state of being inserted into the blood vessel, created based on a fluoroscopic image in a state where the wire is inserted into the blood vessel, and information indicating elasticity of the wire.

(13) The non-transitory computer-readable medium according to any one of (1) to (12), causes the computer to execute processing further including acquiring the blood vessel cross-sectional image representing the cross section of the blood vessel, inputting the acquired blood vessel cross-sectional image to a learned model that outputs the lesion information in a case where the blood vessel cross-sectional image is input, and acquiring the lesion information output by the learned model.

(14) The non-transitory computer-readable medium according to any one of (1) to (13), causes the computer to execute processing further including specifying the recommended device, according to the type and the severity of the lesion portion included in the lesion information and the direction and the strength of pressing the wire against the inner surface of the blood vessel, included in the bias information.

(15) An information processing apparatus according to an embodiment of the present disclosure includes a processor configured to: acquire lesion information indicating a state of a lesion portion included in a blood vessel of a patient and bias information of a wire to be inserted into the blood vessel and specify a recommended device recommended to be used for treatment on the lesion portion, based on the lesion information and the bias information, and device information of a treatment device used for intravascular treatment.

(16) An information processing method according to an embodiment of the present disclosure includes acquiring lesion information indicating a state of a lesion portion included in a blood vessel of a patient and bias information of a wire to be inserted into the blood vessel and specifying a recommended device recommended to be used for treatment on the lesion portion, based on the lesion information and the bias information, and device information of a treatment device used for intravascular treatment.

In one embodiment of the present disclosure, lesion information representing a state of a lesion portion included in a blood vessel of a patient and bias information indicating a wire bias are acquired, and a recommended device is specified based on the lesion information and the bias information, and device information indicating characteristics of a treatment device. A user can easily select an appropriate use device according to the state of the lesion portion, by using the specified recommended device as a use device to be used for intravascular treatment.

According to the present disclosure, it is easier to select an appropriate use device. Therefore, the present disclosure exhibits an excellent effect such that intravascular treatment is stably and appropriately performed.

### BRIEF DESCRIPTION OF THE DRAWINGS

FIG. 1 is a schematic diagram illustrating an outline of a treatment system used to perform intravascular treatment.

FIG. 2 is a block diagram illustrating an internal functional configuration example of an information processing apparatus.

FIG. 3 is a conceptual diagram illustrating a content example of device data.

FIG. 4 is a flowchart illustrating an example of a procedure of processing for assisting selection of a treatment device executed by the treatment system.

FIG. 5 is a flowchart illustrating an example of the procedure of the processing for assisting the selection of the treatment device executed by the treatment system.

FIG. 6 is a schematic diagram illustrating an example of a three-dimensional blood vessel model representing three-dimensional shapes of a blood vessel and a guide wire.

FIG. 7 is a schematic diagram illustrating a first example of a display image displayed by a display device.

FIG. 8 is a schematic diagram illustrating a second example of the display image displayed by the display device.

FIG. 9 is a schematic diagram illustrating an example of a display image in which a treatment range is displayed.

FIG. 10 is a schematic diagram illustrating an example of a display image in which a use device is designated.

FIG. 11 is a schematic diagram illustrating an example of a display image after the intravascular treatment has been performed.

FIG. 12 is a block diagram illustrating an internal functional configuration example of an information processing apparatus using a learned model.

FIG. 13 is a conceptual diagram illustrating functions of the learned model.

### DETAILED DESCRIPTION

Set forth below with reference to the accompanying drawings is a detailed description of embodiments of a non-transitory computer-readable medium storing a computer program, an information processing apparatus, and an information processing method related to intravascular treatment.

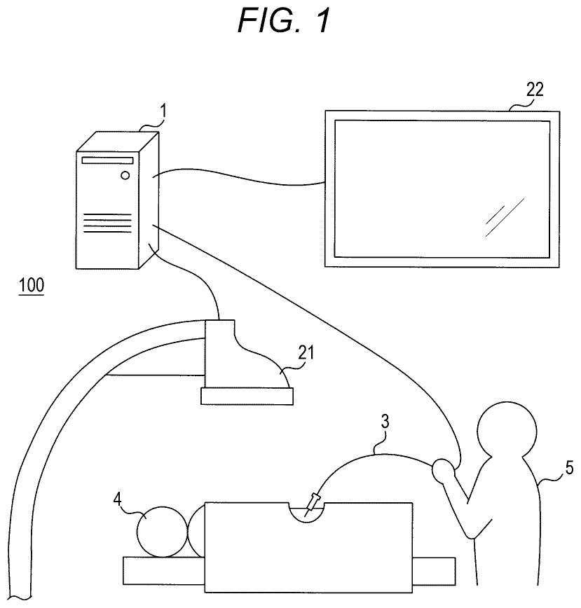

FIG. 1 is a schematic diagram illustrating an outline of a treatment system 100 used to perform intravascular treatment. A patient 4 has a disease in a blood vessel. The blood vessel of the patient 4 includes a lesion portion. The treatment system 100 is a system that performs work for intravascular treatment using a device 3 to be percutaneously inserted into the blood vessel of the patient 4. A user 5 of the treatment system 100 is a practitioner who performs the intravascular treatment.

The treatment system 100 includes an information processing apparatus 1, a fluoroscopic device 21, and a display device 22. The fluoroscopic device 21 generates a fluoroscopic image in which the blood vessel of the patient 4 into which a contrast dye is injected is fluoroscopically viewed from outside of the patient 4. For example, the fluoroscopic device 21 irradiates the patient 4 with an X-ray, detects the X-ray that has passed through the patient 4, and generates the fluoroscopic image in which the blood vessel of the patient 4 that has been fluoroscopically viewed is imaged. Furthermore, the fluoroscopic device 21 changes an imaging direction, fluoroscopically views the blood vessel of the patient 4 from a plurality of directions, and generates a plurality of imaged fluoroscopic images.

The display device 22 displays an image. The display device 22 can be, for example, a liquid crystal display or an electroluminescent (EL) display. The information processing apparatus 1 is connected to the fluoroscopic device 21 and the display device 22. The information processing apparatus 1 acquires the fluoroscopic image generated by the fluoroscopic device 21 and displays the fluoroscopic image on the display device 22. Furthermore, the information processing apparatus 1 executes processing for generating a three-dimensional blood vessel model representing a three-dimensional shape of the blood vessel of the patient 4, based on the plurality of fluoroscopic images generated by the fluoroscopic device 21.

In the intravascular treatment, a plurality of devices 3 can be used. Each device 3 has an elongated shape that can be inserted into a blood vessel. First, a guide wire is used as the device 3. The guide wire is inserted into the blood vessel of the patient 4. Next, a measuring device that measures a cross section of the blood vessel is used as the device 3 in order to generate a blood vessel cross-sectional image representing the cross section of the blood vessel. The blood vessel cross-sectional image is generated by optical coherence tomography (OCT), optical frequency domain imaging (OFDI), or intravascular ultrasound (IVUS). The measuring device has a configuration in which a measuring unit is provided at a distal end of a catheter, and is connected to the information processing apparatus 1. The measuring unit generates or receives light such as infrared light or generates or detects ultrasonic waves. Hereinafter, the generation or the reception of light or the generation or the detection of the ultrasonic waves performed by the measuring unit is referred to as measurement.

The measuring device is inserted into the blood vessel of the patient 4, along the guide wire. Data representing the light received by the measuring unit or data representing the ultrasonic waves detected by the measuring unit is input to the information processing apparatus 1. The information processing apparatus 1 generates the blood vessel cross-sectional image representing the cross section of the blood vessel, based on the data input from the measuring device. The measuring device measures a plurality of points in the blood vessel, and the information processing apparatus 1 generates the plurality of blood vessel cross-sectional images representing the cross sections at the plurality of points of the blood vessel of the patient 4. The information processing apparatus 1 displays the blood vessel cross-sectional image on the display device 22.

Next, a treatment device that performs work for treatment in a blood vessel is used as the device 3. There are various types of treatment devices such as an atherectomy device, a thrombectomy device, or a thrombus aspiration catheter. An appropriate treatment device according to the lesion portion of the blood vessel may be used. The treatment device is inserted into the blood vessel of the patient 4, along the guide wire. The measuring device is withdrawn from the blood vessel of the patient 4, and instead, the treatment device is inserted into the blood vessel. Alternatively, the treatment device may be inserted into the blood vessel of the patient 4 together with the measuring device. The user 5 operates the treatment device while visually recognizing the fluoroscopic image displayed on the display device 22 and performs the intravascular treatment.

For appropriate intravascular treatment, it is important to use an appropriate treatment device according to the lesion portion of the blood vessel. The treatment system 100 executes processing for assisting selection of the treatment device used for the intravascular treatment. The information processing apparatus 1 executes an information processing method for assisting the selection of the treatment device.

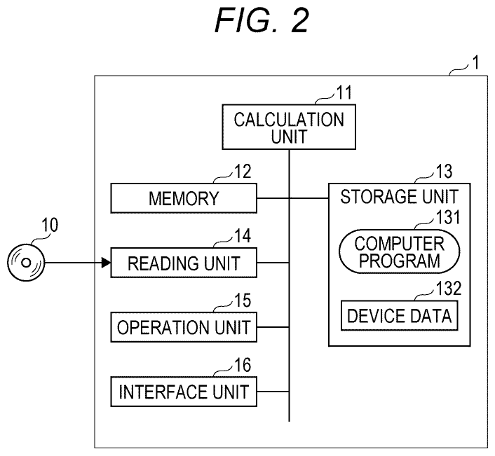

FIG. 2 is a block diagram illustrating an internal functional configuration example of the information processing apparatus 1. The information processing apparatus 1 can be, for example, a computer such as a server device or a personal computer. The information processing apparatus 1 includes a calculation unit 11, a memory 12, a storage unit 13, a reading unit 14, an operation unit 15, and an interface unit 16. The calculation unit 11 can be configured, for example, using a central processing unit (CPU), a graphics processing unit (GPU), or a multi-core CPU. The calculation unit 11 may be configured using a quantum computer. The memory 12 stores temporary data generated in association with calculation. The memory 12 can be, for example, a random access memory (RAM). The storage unit 13 can be nonvolatile and can be, for example, a non-transitory computer-readable medium such as a hard disk or a nonvolatile semiconductor memory. The reading unit 14 can read information from a recording medium (or non-transitory computer-readable medium) 10 such as an optical disk or a portable memory.

The calculation unit 11 causes the reading unit 14 to read a computer program 131 recorded in the recording medium 10 and causes the storage unit 13 to store the read computer program 131. The calculation unit 11 executes processing necessary for the information processing apparatus 1, according to the computer program 131. The computer program 131 may be a computer program product. The computer program 131 may be downloaded from outside of the information processing apparatus 1. Alternatively, the computer program 131 may be stored in the information processing apparatus 1 in advance. In these cases, the information processing apparatus 1 does not need to include the reading unit 14.

The operation unit 15 receives an input of information such as text by receiving an operation from the user 5. The operation unit 15 can be, for example, a touch panel, a pen tablet, a keyboard, or a pointing device. The interface unit 16 is connected to the fluoroscopic device 21 and the display device 22, and is connected to the measuring device. The fluoroscopic device 21 transmits the generated fluoroscopic image to the information processing apparatus 1, and the information processing apparatus 1 receives the fluoroscopic image transmitted from the fluoroscopic device 21 with the interface unit 16. The information processing apparatus 1 transmits the image from the interface unit 16 to the display device 22, and the display device 22 displays the image received from the information processing apparatus 1. The information processing apparatus 1 receives the data input from the measuring device, using the interface unit 16. Note that, the operation unit 15 may be provided outside the information processing apparatus 1 and connected to the interface unit 16.

The information processing apparatus 1 may include a plurality of computers, and data may be distributed and stored in the plurality of computers, and processing may be distributed and executed by the plurality of computers. The information processing apparatus 1 may be implemented by using cloud computing or may be implemented by a plurality of virtual machines provided in a single computer.

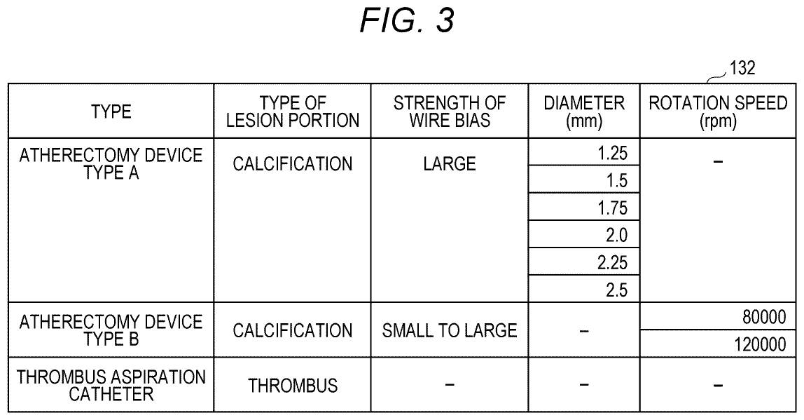

The storage unit 13 stores device data 132 in which information regarding the plurality of treatment devices is recorded. FIG. 3 is a conceptual diagram illustrating a content example of the device data 132. In the device data 132, device information indicating characteristics of each of the plurality of treatment devices is recorded. In FIG. 3, an example is illustrated in which a type A of an atherectomy device, a type B of an atherectomy device, and a thrombus aspiration catheter are recorded as types of the treatment device.

The type A of the atherectomy device is a device that includes a drill (or drill-like tool) and cuts the lesion portion of the blood vessel from the inside of the blood vessel. The type B of the atherectomy device is a device that cuts, from the inside of the blood vessel, a lesion portion in a larger range than a range cut by the type A, by rotating an orbital of a cutting member provided in the middle of the catheter. The thrombus aspiration catheter is a device that aspirates a thrombus in a case where the lesion portion is the thrombus. In the device data 132, a type of the lesion portion to be a target of the intravascular treatment using each treatment device is recorded, in association with the type of the treatment device. A type of the lesion portion to be the target of the intravascular treatment using the types A and B of the atherectomy device is calcification. In a case where the lesion portion is calcified, it is appropriate to cut the lesion portion using the atherectomy device of the type A or the type B. A type of the lesion portion to be the target of the intravascular treatment using the thrombus aspiration catheter is a thrombus.

In the device data 132, an appropriate strength of a wire bias when each treatment device is used is recorded, in association with the type of the treatment device. The wire bias is a phenomenon in which the guide wire inserted into the blood vessel of the patient 4 is pressed against an inner surface of the blood vessel. A direction of the wire bias is a direction in which the guide wire is pressed against the inner surface of the blood vessel, and a strength of the wire bias is a magnitude of a force of the guide wire pressed against the inner surface of the blood vessel. In the example illustrated in FIG. 3, the type A of the atherectomy device is associated with that an appropriate strength of the wire bias is large. It is indicated that it is appropriate to use the type A of the atherectomy device, in a case where the strength of the wire bias is large. Furthermore, the type B of the atherectomy device is associated with a strength of the wire bias from small to large. It is indicated that the atherectomy device of the type B can be used regardless of whether the strength of the wire bias is large or small.

In the device data 132, a diameter of the drill (or drill-like tool) included the atherectomy device of the type A is recorded, in association with the type A of the atherectomy device. There are types A of the atherectomy device having different diameters of the drill, and each of the diameters is recorded. Depending on the diameter of the drill, a range where the lesion portion is cut differs. In the device data 132, a rotation speed of the orbital rotation of the type B of the atherectomy device is recorded, in association to the type B of the atherectomy device. In FIG. 3, a unit of the rotation speed is set as rotations per minute (rpm). There are types B of the atherectomy device having different rotation speeds, and each rotation speed is recorded. Depending on the rotation speed, the range where the lesion portion is cut differs. The type of the treatment device recorded in the device data 132 and the information recorded in association with the type of the treatment device are the device information indicating the characteristics of each treatment device.

In the device data 132, device information indicating characteristics of the treatment device other than the characteristics illustrated in FIG. 3 may be recorded. In the device data 132, as the type of the treatment device, a device other than the type A of the atherectomy device, the type B of the atherectomy device, and the thrombus aspiration catheter may be recorded.

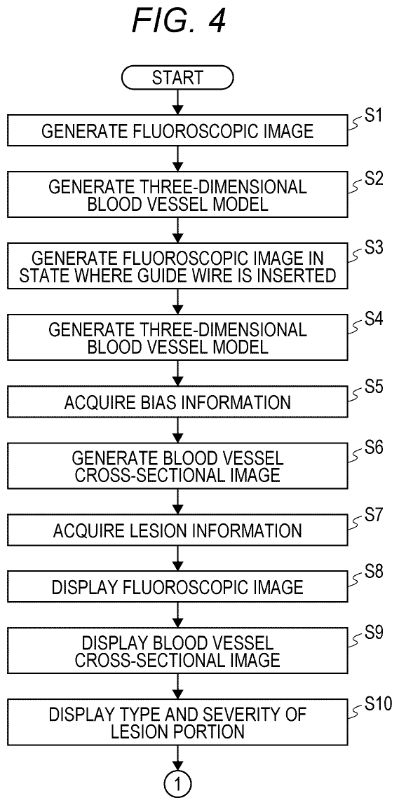

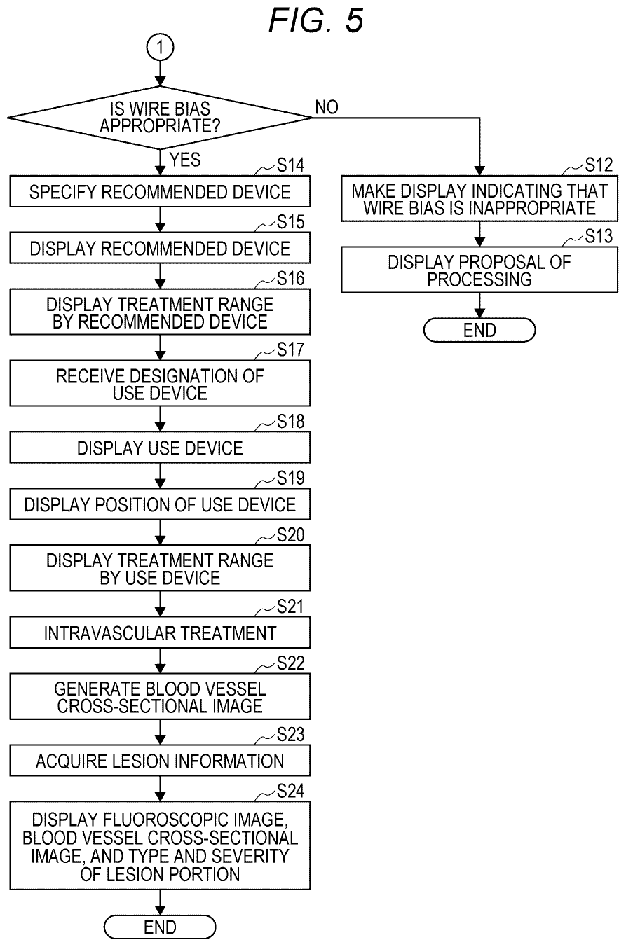

Processing executed by the treatment system 100 will be described. FIGS. 4 and 5 are flowcharts illustrating an example of a procedure of the processing for assisting the selection of the treatment device executed by the treatment system 100. Hereinafter, a step is abbreviated as S. The calculation unit 11 executes information processing according to the computer program 131, and the information processing apparatus 1 executes the following processing. In a state where the contrast dye is injected into the blood vessel of the patient 4, the treatment system 100 generates the fluoroscopic image in which the blood vessel of the patient 4 is fluoroscopically viewed (S1). In S1, the fluoroscopic device 21 fluoroscopically views the blood vessel of the patient 4 from the plurality of directions with X rays, generates the plurality of fluoroscopic images in which the blood vessel of the patient 4 is imaged from the plurality of directions, and inputs the plurality of generated fluoroscopic images to the information processing apparatus 1. The information processing apparatus 1 acquires the plurality of fluoroscopic images by receiving input of the fluoroscopic images from the fluoroscopic device 21 and stores the fluoroscopic images in the storage unit 13.

The information processing apparatus 1 generates the three-dimensional blood vessel model representing the three-dimensional shape of the blood vessel of the patient 4 (S2). In S2, the calculation unit 11 generates the three-dimensional blood vessel model representing the three-dimensional shape of the blood vessel of the patient 4, based on the plurality of fluoroscopic images imaged from the plurality of directions. The calculation unit 11 stores the generated three-dimensional blood vessel model in the storage unit 13. In S1 and S2, a technique of rotational angiography is used.

After the processing in S1 or S2 is executed, the user 5 inserts the guide wire into the blood vessel of the patient 4. In a state where the guide wire is inserted into the blood vessel, the treatment system 100 generates the fluoroscopic image in which the blood vessel of the patient 4 is fluoroscopically viewed (S3). In S3, the fluoroscopic device 21 generates the plurality of fluoroscopic images obtained by imaging the blood vessel in which the guide wire is inserted, from the plurality of directions, through the processing as in S1, and the information processing apparatus 1 acquires the plurality of fluoroscopic images and stores the fluoroscopic images in the storage unit 13. In the fluoroscopic image generated in S3, the guide wire inserted into the blood vessel is imaged.

The information processing apparatus 1 generates the three-dimensional blood vessel model (S4). In S4, the calculation unit 11 generates the three-dimensional blood vessel model based on the three-dimensional blood vessel model generated in S2 and the plurality of fluoroscopic images acquired in S3. The calculation unit 11 stores the generated three-dimensional blood vessel model in the storage unit 13. The three-dimensional blood vessel model generated in S4 represents three-dimensional shapes of the blood vessel of the patient 4 and the guide wire in the blood vessel.

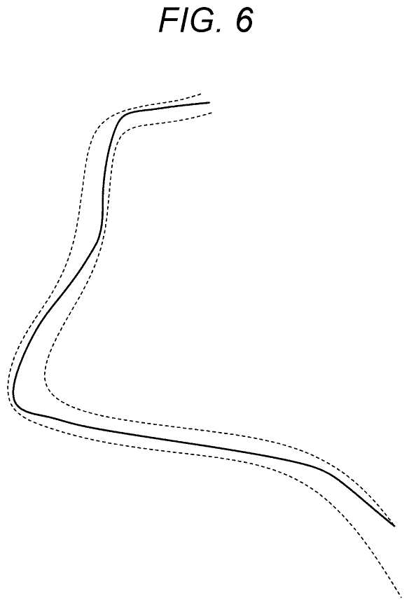

FIG. 6 is a schematic diagram illustrating an example of the three-dimensional blood vessel model representing the three-dimensional shapes of the blood vessel and the guide wire. In FIG. 6, the inner surface of the blood vessel is indicated by a broken line, and the guide wire is indicated by a solid line. The guide wire is inserted along the inner surface of the blood vessel. The blood vessel is tortuous, and the guide wire is inserted while being deformed. It is not possible to deform the guide wire in a shape exactly the same as the blood vessel. Therefore, a distance between the inner surface of the blood vessel and the guide wire varies depending on a position. Depending on the position in the blood vessel, the wire bias that is the phenomenon in which the guide wire is pressed against the inner surface of the blood vessel occurs. Since the three-dimensional blood vessel model represents the three-dimensional shapes of the blood vessel and the guide wire, a state of the wire bias at each position in the blood vessel is clarified from the three-dimensional blood vessel model.

The information processing apparatus 1 acquires bias information indicating the wire bias (S5). In S5, the calculation unit 11 acquires the bias information including a direction and a strength of the wire bias, by specifying a direction and a strength of the wire bias at each position of the blood vessel, based on the three-dimensional blood vessel model. The direction of the wire bias is represented, for example, by a clock position centered on the center of the blood vessel in a cross section perpendicular to a length direction of the blood vessel. The calculation unit 11 specifies the direction of the wire bias, based on a positional relationship between the inner surface of the blood vessel and the guide wire on the cross section at each position of the blood vessel. The positional relationship between the inner surface of the blood vessel and the guide wire is obtained from the three-dimensional blood vessel model. For example, in a case where the guide wire is deviated from the center of the blood vessel in the cross section, a direction of a portion of the inner surface closest to the guide wire viewed from the center of the blood vessel is the direction of the wire bias.

The strength of the wire bias is a magnitude of the force of the guide wire being pressed against the inner surface of the blood vessel. The calculation unit 11 specifies the strength of the wire bias, based on the distance between the inner surface of the blood vessel and the guide wire at each position of the blood vessel, the shape of the guide wire at each position, and elasticity of the guide wire. The distance between the inner surface of the blood vessel and the guide wire and the shape of the guide wire at each position are obtained from the three-dimensional blood vessel model. Information indicating the elasticity of the wire bias is stored in the storage unit 13 in advance or is input by operating the operation unit 15 by the user 5. For example, when the distance between the inner surface of the blood vessel and the guide wire is shorter, a bending degree of the guide wire is larger, and the elasticity of the guide wire is larger, the strength of the wire bias is larger. The calculation unit 11 stores the bias information at the plurality of positions in the blood vessel, in the storage unit 13. The processing in S5 corresponds to an acquisition unit.

After the processing in S4 or S5 is executed, the user 5 inserts the measuring device into the blood vessel of the patient 4. The treatment system 100 generates the blood vessel cross-sectional image representing the cross section of the blood vessel of the patient 4, using the measuring device (S6). In S6, the measuring device performs measurement at a plurality of positions while moving in the blood vessel along the guide wire, and inputs the data into the information processing apparatus 1. The information processing apparatus 1 generates the blood vessel cross-sectional images at the plurality of positions of the blood vessel, based on the data from the measuring device. The calculation unit 11 stores the plurality of blood vessel cross-sectional images in the storage unit 13. Furthermore, the calculation unit 11 stores each position in the blood vessel where the blood vessel cross-sectional image is generated, in the storage unit 13.

The information processing apparatus 1 acquires lesion information indicating a state of a lesion portion included in the blood vessel of the patient 4 (S7). In S7, the calculation unit 11 acquires the lesion information including a type and a severity of the lesion portion, by specifying a type and a severity of the lesion portion included in the blood vessel, based on the blood vessel cross-sectional image. The type of the lesion portion can include calcification, a thrombus, or the like. The severity of the lesion portion indicates a degree of seriousness of the lesion portion. For example, the severity of the lesion portion is represented by a score in which the degree of the seriousness increases as a numerical value increases.

In S7, the calculation unit 11 determines absence or presence of the lesion portion at each position of the blood vessel, by executing image processing on the blood vessel cross-sectional image at each position of the blood vessel. For example, the calculation unit 11 determines a lumen of the blood vessel, a wall of the blood vessel, and the lesion portion, according to a luminance distribution in the blood vessel cross-sectional image. Furthermore, the calculation unit 11 determines the type of the lesion portion, according to the luminance distribution of the lesion portion in the blood vessel cross-sectional image. For example, in a case where there is a clear boundary between a luminance of the lesion portion and a luminance of the wall of the blood vessel, the calculation unit 11 determines that the type of the lesion portion is calcification. For example, in a case where there is no clear boundary between the luminance of the lesion portion and the luminance of the wall of the blood vessel, the calculation unit 11 determines that the type of the lesion portion is a thrombus.

Furthermore, the calculation unit 11 specifies a position of the lesion portion, according to the position in the blood vessel where each blood vessel cross-sectional image is obtained. The calculation unit 11 determines a length of the lesion portion along the length direction of the blood vessel, based on the position of the lesion portion. The calculation unit 11 specifies the severity of the lesion portion, according to a size and a shape of the lesion portion and a length of the lesion portion in the blood vessel cross-sectional image. For example, as the size of the lesion portion increases, the severity of the lesion portion increases. The position, the size, the shape, and the length of the lesion portion may be included in the lesion information. The calculation unit 11 stores the lesion information in the storage unit 13. In a case where there is the plurality of lesion portions, the calculation unit 11 may acquire the lesion information for each of the plurality of lesion portions and store the lesion information in the storage unit 13. The processing in S7 corresponds to the acquisition unit.

The information processing apparatus 1 displays the fluoroscopic image of the blood vessel of the patient 4 on the display device 22 (S8). In S8, the calculation unit 11 transmits the fluoroscopic image from the interface unit 16 to the display device 22, and displays the fluoroscopic image on the display device 22. The displayed fluoroscopic image is an image generated by the fluoroscopic device 21 and stored in the storage unit 13. The calculation unit 11 may reconstruct the fluoroscopic image from the three-dimensional blood vessel model and display the reconstructed fluoroscopic image on the display device 22.

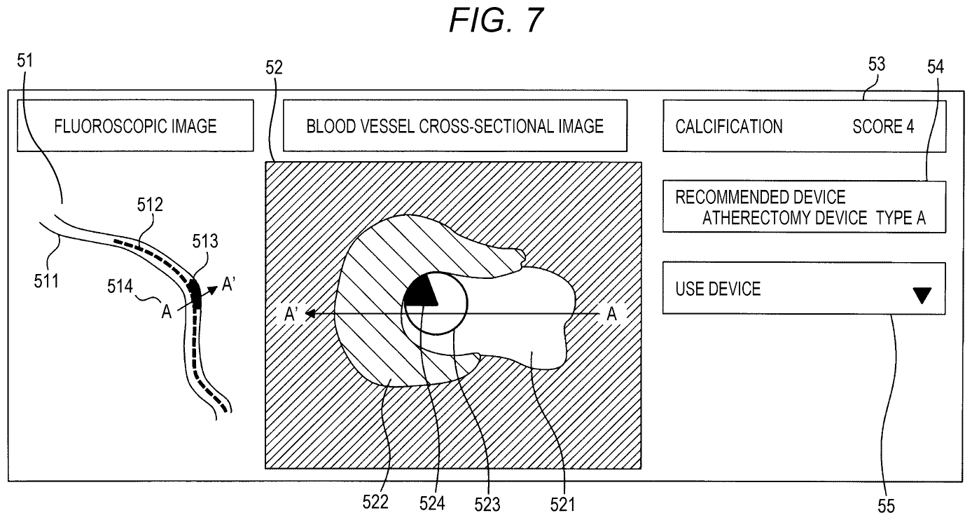

FIG. 7 is a schematic diagram illustrating a first example of a display image displayed by the display device 22. The display image includes a fluoroscopic image 51. The fluoroscopic image 51 includes a blood vessel 511. In FIG. 7, the blood vessel 511 is indicated by a solid line. The information processing apparatus 1 superimposes a trajectory 512 of the plurality of positions in the blood vessel where the blood vessel cross-sectional images are created on the fluoroscopic image 51 and displays the trajectory 512 on the display device 22. At this time, the calculation unit 11 executes processing for generating an image of the trajectory 512 based on each position in the blood vessel where the blood vessel cross-sectional image is created and displaying the image of the trajectory 512 on the display device 22. In FIG. 7, the trajectory 512 is indicated by a broken line. By displaying the trajectory 512, the user 5 can confirm which route in the blood vessel the device 3 passes through and on which route the blood vessel cross-sectional image is generated.

Furthermore, the information processing apparatus 1 superimposes a position 513 of the lesion portion on the fluoroscopic image 51 and displays the position 513 on the display device 22. At this time, the calculation unit 11 generates an image indicating the position 513 of the lesion portion, according to the position of the lesion portion included in the lesion information stored in the storage unit 13 and displays the image indicating the position 513 of the lesion portion on the display device 22. By displaying the position 513 of the lesion portion, the user 5 can confirm a position in the blood vessel where the lesion portion exists.

The information processing apparatus 1 displays the blood vessel cross-sectional image on the display device 22 (S9). In S9, the calculation unit 11 transmits the blood vessel cross-sectional image from the interface unit 16 to the display device 22 and displays the blood vessel cross-sectional image on the display device 22. The blood vessel cross-sectional image to be displayed is an image stored in the storage unit 13. As illustrated in FIG. 7, a blood vessel cross-sectional image 52 is included in the display image, and the fluoroscopic image 51 and the blood vessel cross-sectional image 52 are displayed at the same time.

The information processing apparatus 1 superimposes a position 514 where the displayed blood vessel cross-sectional image 52 is created on the fluoroscopic image 51 and displays the position 514. At this time, the calculation unit 11 executes processing for generating an image indicating the position 514 of the blood vessel cross-sectional image 52, based on the position in the blood vessel where the blood vessel cross-sectional image 52 is created and displaying the image indicating the position 514 on the display device 22. By displaying the position 514 of the blood vessel cross-sectional image 52, the user 5 can confirm a cross section at which position of the blood vessel is displayed as the blood vessel cross-sectional image 52.

The blood vessel cross-sectional image 52 can include a lumen 521 of the blood vessel, a lesion portion 522, and a guide wire 523. For example, a cross section of the guide wire 523 is included in the blood vessel cross-sectional image 52. Furthermore, the information processing apparatus 1 superimposes and displays the direction and the strength of the wire bias on the blood vessel cross-sectional image 52. At this time, the calculation unit 11 executes processing for generating an image indicating the direction and the strength of the wire bias, based on the bias information and displaying an image 524 indicating the direction and the strength of the wire bias, on the display device 22. In FIG. 7, the image 524 indicating the direction and the strength of the wire bias is a fan-shaped figure spreading from a center position of the guide wire 523. The direction of the wire bias is indicated as a spreading direction of the fan-shaped figure from the center position of the guide wire 523. The strength of the wire bias is represented by a color of the fan-shaped figure. For example, the higher the strength of the wire bias is, the darker the color of the fan-shaped figure is. The image 524 indicating the direction and the strength of the wire bias may be an image other than the fan-shaped figure. The user 5 can rather easily confirm the direction and the strength of the wire bias.

The information processing apparatus 1 displays the type and the severity of the lesion portion on the display device 22 (S10). In S10, the calculation unit 11 executes processing for generating an image indicating the type and the severity of the lesion portion, based on the lesion information and displaying the image indicating the type and the severity of the lesion portion on the display device 22. As illustrated in FIG. 7, an image 53 indicating the type and the severity of the lesion portion is included in the display image. The image 53 indicating the type and the severity of the lesion portion is displayed simultaneously with the fluoroscopic image 51 and the blood vessel cross-sectional image 52. In FIG. 7, an example is illustrated in which the type of the lesion portion is calcification, and the severity of the lesion portion is a “SCORE 4”. The user 5 can confirm the type and the severity of the lesion portion.

The information processing apparatus 1 determines whether or not the wire bias is appropriate for performing the intravascular treatment using the treatment device (S11). In S11, the calculation unit 11 determines whether or not the direction of the wire bias corresponds to the position of the lesion portion. In a state where the direction of the wire bias corresponds to the position of the lesion portion, the guide wire is pressed against the lesion portion. In this state, the treatment device is pressed against the lesion portion, and the lesion portion is affected by an operation of the treatment device. In a state where the direction of the wire bias does not correspond to the position of the lesion portion, the guide wire is not pressed against the lesion portion. In this state, the treatment device is not pressed against the lesion portion, and the lesion portion is not affected even if the treatment device is operated. In a case where the direction of the wire bias does not correspond to the position of the lesion portion, the calculation unit 11 determines that the wire bias is inappropriate.

Furthermore, in a case where the direction of the wire bias corresponds to the position of the lesion portion, the calculation unit 11 determines whether or not the strength of the wire bias matches the appropriate strength of the wire bias included in the device information recorded in the device data 132. In a case where the strength of the wire bias does not match the appropriate strength of the wire bias, appropriate treatment is not performed even if the treatment device is operated. In a case where the strength of the wire bias does not match the appropriate strength of the wire bias, the calculation unit 11 determines that the wire bias is inappropriate. In a case where the strength of the wire bias matches the appropriate strength of the wire bias, the calculation unit 11 determines that the wire bias is appropriate.

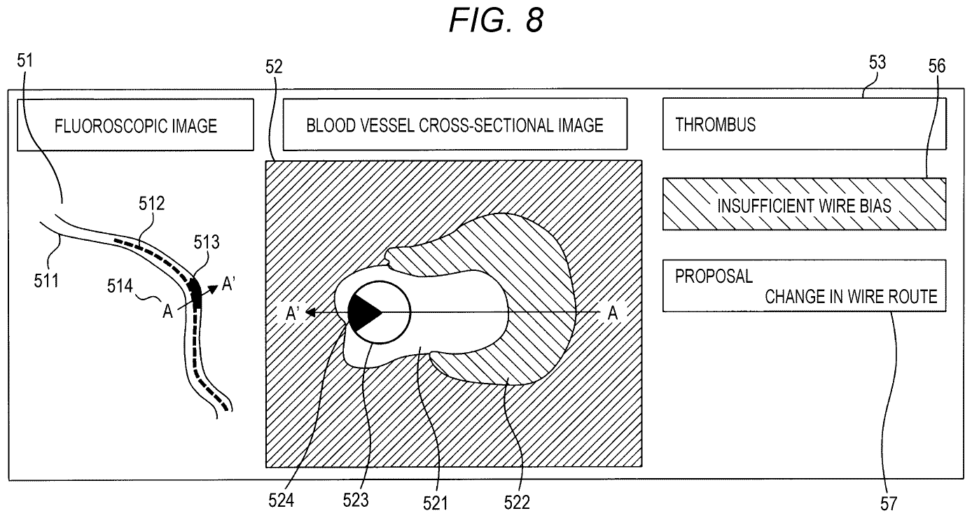

FIG. 8 is a schematic diagram illustrating a second example of the display image displayed by the display device 22. In FIG. 8, an example is illustrated in which the wire bias is inappropriate. The lesion portion 522 does not exist at a position corresponding to the direction of the wire bias indicated by the image 524 included in the display image.

In a case where the wire bias is inappropriate (S11: No), the information processing apparatus 1 makes a display indicating that the wire bias is inappropriate on the display device 22 (S12). In S12, the calculation unit 11 executes processing for generating an image indicating that the wire bias is inappropriate and displaying the image indicating that the wire bias is inappropriate on the display device 22. A case where the wire bias is inappropriate is a case where the bias information is not suitable for the intravascular treatment. As illustrated in FIG. 8, an image 56 indicating that the wire bias is inappropriate is included in the display image. The image 56 indicating that the wire bias is inappropriate is displayed simultaneously with the fluoroscopic image 51 and the blood vessel cross-sectional image 52.

In a case where the wire bias is inappropriate, even if the intravascular treatment using the treatment device is performed, it is not possible to expect an effect of the treatment. Therefore, it is not possible to perform effective intravascular treatment, and it is not necessary to designate the treatment device to be used for the intravascular treatment. By making the display indicating that the wire bias is inappropriate, the user 5 can confirm that it is not necessary to designate the treatment device.

Next, the information processing apparatus 1 displays a proposal of processing for enabling the intravascular treatment on the display device 22 (S13). In S13, the calculation unit 11 executes processing for specifying the proposal of the processing for enabling the intravascular treatment and displaying an image indicating the specified proposal on the display device 22. For example, a table in which the bias information indicating the inappropriate wire bias and content of the proposal are associated is stored, and the calculation unit 11 reads the content of the processing according to the bias information from the table, so as to specify the proposal of the processing.

As illustrated in FIG. 8, an image 57 indicating the proposal of the processing is included in the display image. The content of the proposal of the processing includes a change in a wire route that is a route in the blood vessel through which the guide wire passes or a change in the guide wire. For example, in a case where the direction of the wire bias does not correspond to the position of the lesion portion, the change in the wire route is proposed. For example, in a case where the strength of the wire bias is insufficient, the change in the guide wire is proposed. By executing the processing according to the proposal by the user 5, conditions of the blood vessel and the guide wire change, the wire bias changes, and there is a possibility that the effective intravascular treatment can be performed.

After S13 ends, the information processing apparatus 1 ends the processing. Thereafter, the user 5 may execute the proposed processing such as the change in the wire route or the change in the guide wire. Moreover, thereafter, the processing in S3 and subsequent steps may be executed again.

In a case where the wire bias is appropriate (S11: YES), the information processing apparatus 1 specifies a recommended device that is a treatment device recommended to be used for treatment on the lesion portion, based on the device information, the lesion information, and the bias information (S14). In S14, the calculation unit 11 specifies the recommended device, by selecting any one treatment device from among the plurality of treatment devices of which the device information is recorded in the device data 132. For example, the calculation unit 11 selects a treatment device of which a type of a lesion portion to be the target of the intravascular treatment included in device information matches the type of the lesion portion included in the lesion information. Furthermore, the calculation unit 11 further selects a treatment device of which an appropriate strength of a wire bias when the treatment device is used included in the device information matches the strength of the wire bias included in the bias information, from among the selected treatment devices. In this way, the recommended device can be specified. The processing in S14 corresponds to a specification unit.

The information processing apparatus 1 displays the specified recommended device on the display device 22 (S15). In S15, the calculation unit 11 executes processing for generating an image indicating the recommended device and displaying the image indicating the recommended device on the display device 22. As illustrated in FIG. 7, an image 54 indicating the recommended device is included in the display image. The image 54 indicating the recommended device can be displayed simultaneously with the fluoroscopic image 51 and the blood vessel cross-sectional image 52. In FIG. 7, an example is illustrated in which the image 54 indicating a type of the recommended device as characters is displayed. The user 5 can easily confirm the recommended device.

The information processing apparatus 1 displays an expected treatment range by the recommended device, on the display device 22 (S16). The treatment range is a treatable region in the lesion portion in a case where the recommended device operates as the treatment device. In S16, the calculation unit 11 specifies the expected treatment range, based on the device information associated with the recommended device, generates an image indicating the treatment range, superimposes the image indicating the treatment range on the blood vessel cross-sectional image 52, and displays the image on the display device 22. In a case where the recommended device is the type A of the atherectomy device, the treatment range is specified according to the diameter of the drill (or drill-like tool) included in the device information. In a case where the recommended device is the type B of the atherectomy device, the treatment range is specified according to the rotation speed included in the device information.

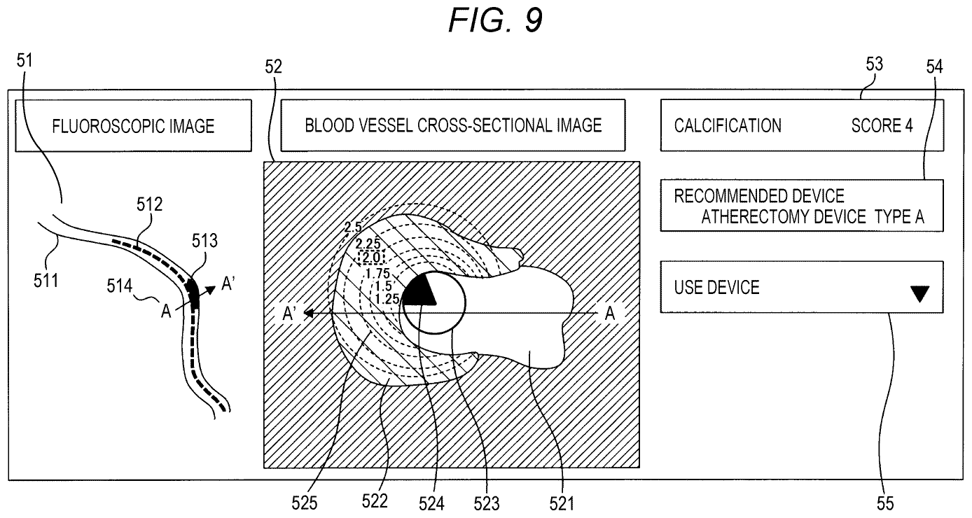

FIG. 9 is a schematic diagram illustrating an example of the display image in which the treatment range is displayed. In FIG. 9, an example is illustrated in which the recommended device is the atherectomy device of the type A. The treatment range is a range cut by the type A of the atherectomy device. The range to be cut is determined according to the diameter of the atherectomy device of the type A. For example, a diameter of the range to be cut is the same as the diameter of the atherectomy device of the type A. The calculation unit 11 specifies the treatment range according to each diameter included in the device information and displays the treatment range on the display device 22. In FIG. 9, a treatment range 525 according to each diameter is indicated by a broken line. The user 5 can confirm how large the treatment range based on the diameter of the drill of the atherectomy device of the type A.

In a case where the recommended device is the type B of the atherectomy device, the treatment range is a range cut by the atherectomy device of the type B. The range to be cut is determined according to the rotation speed of the atherectomy device of the type B. The calculation unit 11 specifies the treatment range according to each rotation speed included in the device information and displays the treatment range on the display device 22. In this case, the treatment range according to each rotation speed is superimposed and displayed on the blood vessel cross-sectional image 52. In a case where the recommended device is an aspiration catheter, the treatment range is a range aspirated by the aspiration catheter.

In a case where the treatment range is small, a treatment effect can be small. In a case where the treatment range is large, although the treatment effect increases, a risk that the blood vessel of the patient 4 is damaged can increase. The user 5 can select the use device that is the treatment device to be used, after confirming the treatment range and confirming the magnitude of the treatment effect.

The information processing apparatus 1 receives the designation of the use device that is the treatment device to be used (S17). In S17, the operation of the operation unit 15 by the user 5 makes the calculation unit 11 receive the designation of the use device. For example, the type of the treatment device is designated, and in addition, a single treatment device is designated as the use device from among the plurality of treatment devices having different treatment ranges. For example, the type A of the atherectomy device is designated, and a single diameter is designated from among the plurality of diameters. For example, the atherectomy device of the type B is designated, and a single rotation speed is designated from among the plurality of rotation speeds. For example, a single recommended device can be designated as the use device, from among the plurality of recommended devices having the different treatment ranges. A treatment device other than the recommended device may be designated as the use device.

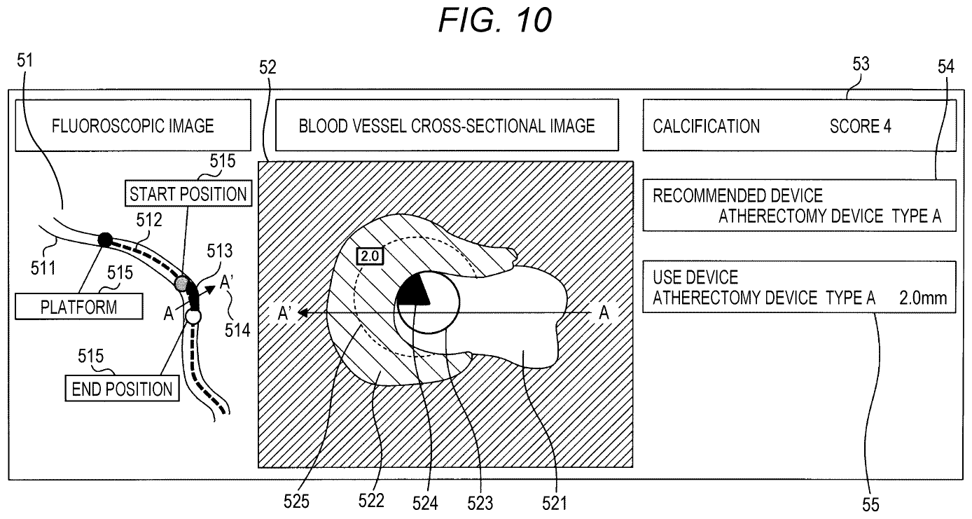

The information processing apparatus 1 displays the use device on the display device 22 (S18). In S18, the calculation unit 11 executes processing for generating an image indicating the designated use device and displaying the image indicating the use device on the display device 22. FIG. 10 is a schematic diagram illustrating an example of a display image in which the use device is designated. An image 55 indicating the use device is included in the display image. In FIG. 10, an example is illustrated in which the use device is the atherectomy device of the type A having a diameter of 2.0 mm and the image 55 indicating the use device as characters is displayed. The image 55 indicating the use device is displayed simultaneously with the fluoroscopic image 51 and the blood vessel cross-sectional image 52. The user 5 can confirm the designated use device.

The information processing apparatus 1 superimposes a position of the use device in the blood vessel when the use device is used on the fluoroscopic image 51 and displays the position on the display device 22 (S19). For example, a length of each portion of the treatment device and a relative position of each portion are included in the device information recorded in the device data 132. In S19, the calculation unit 11 specifies the position of the use device in the blood vessel when the use device is used, based on the position of the lesion portion included in the lesion information, the device information regarding the use device, and the three-dimensional blood vessel model. The calculation unit 11 superimposes an image indicating the specified position of the use device on the fluoroscopic image 51 and displays the image on the display device 22.

As illustrated in FIG. 10, a position 515 of the use device in the blood vessel is superimposed and displayed on the fluoroscopic image 51. In the example illustrated in FIG. 10, a start position and an end position when the use device is used and a position of a platform included in the use device are displayed as the positions 515 of the use device. The start position and the end position are specified from the position of the lesion portion. The position of the platform is specified from a relative position of the platform in the use device. The user 5 can recognize a situation when the use device is used.

The information processing apparatus 1 superimposes a predicted treatment range by the use device on the blood vessel cross-sectional image 52 and displays the treatment range on the display device 22 (S20). The calculation unit 11 executes processing for specifying the treatment range according to the device information of the use device and displaying the treatment range on the display device 22. In FIG. 10, the treatment range 525 by the use device is superimposed on the blood vessel cross-sectional image 52. The user 5 can confirm the treatment range by the use device.

Next, the treatment system 100 executes processing for performing the intravascular treatment (S21). The user 5 performs work for inserting the treatment device that is the use device into the blood vessel of the patient 4. The fluoroscopic device 21 generates the fluoroscopic image, and the information processing apparatus 1 displays the fluoroscopic image on the display device 22. The user 5 performs the work while checking the fluoroscopic image. The treatment device operates according to the operation of the user, and the intravascular treatment for cutting the lesion portion or the like is performed.

After the intravascular treatment, the user 5 inserts the measuring device into the blood vessel of the patient 4. The treatment system 100 generates the blood vessel cross-sectional image, using the measuring device (S22). As in S6, the information processing apparatus 1 generates the blood vessel cross-sectional images at the plurality of positions in the blood vessel and stores the plurality of blood vessel cross-sectional images in the storage unit 13. The information processing apparatus 1 acquires the lesion information indicating the state of the lesion portion included in the blood vessel of the patient 4 (S23). In S23, the calculation unit 11 acquires the lesion information including the type and the severity of the lesion portion, by specifying the type and the severity of the lesion portion, as in S7. Note that a fluoroscopic image may be newly generated.

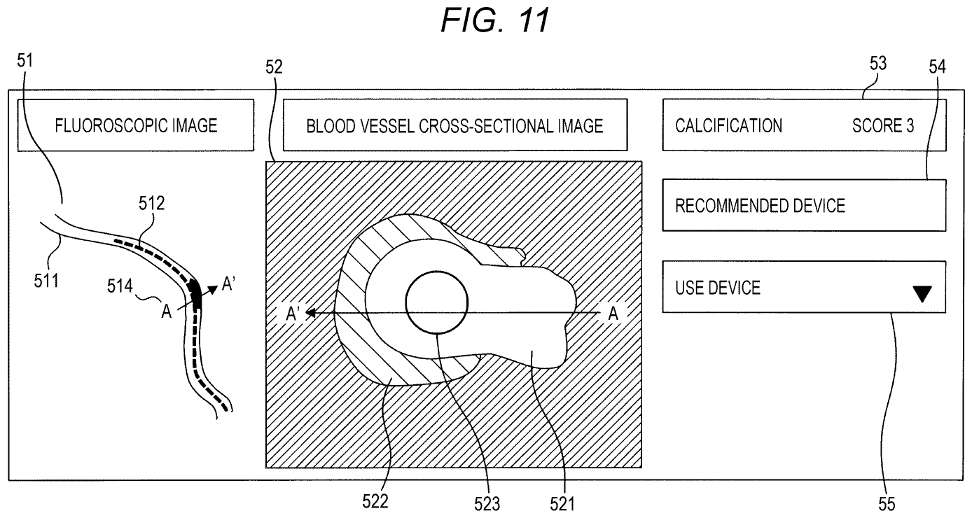

The information processing apparatus 1 displays the fluoroscopic image, the blood vessel cross-sectional image, the type and the severity of the lesion portion on the display device 22 (S24). In S24, the calculation unit 11 executes processing for displaying the fluoroscopic image, the blood vessel cross-sectional image generated in S22, and the type and the severity of the lesion portion according to the lesion information acquired in S23, on the display device 22. FIG. 11 is a schematic diagram illustrating an example of a display image after the intravascular treatment is performed. The lesion portion 522 after the intravascular treatment is performed is included in the blood vessel cross-sectional image 52, and a shape of the lesion portion 522 is changed. Furthermore, the image 53 indicating the type and the severity of the lesion portion is included in the display image, and the severity of the lesion portion is changed. The user 5 can confirm a result of the intravascular treatment.

After S24 ends, the information processing apparatus 1 ends the processing. In a case where the intravascular treatment is insufficient, the processing in S11 and subsequent steps may be executed again. In a case where a lesion portion that is not treated remains in the blood vessel of the patient 4, the position of the blood vessel of which the blood vessel cross-sectional image is displayed is changed, and the processing in S8 and subsequent steps may be executed again.

As described above, the treatment system 100 generates the three-dimensional blood vessel model and the blood vessel cross-sectional image, acquires the bias information and the lesion information, and specifies the recommended device based on the lesion information, the bias information, and the device information. An appropriate recommended device according to the state of the lesion portion is specified based on the lesion information, the bias information, and the device information. By using the recommended device as the use device, the user 5 can easily select an appropriate use device according to the state of the lesion portion. For example, even the user 5 who has relatively little experience can select an appropriate use device. The appropriate treatment device is constantly used, and the intravascular treatment is stably and appropriately performed.

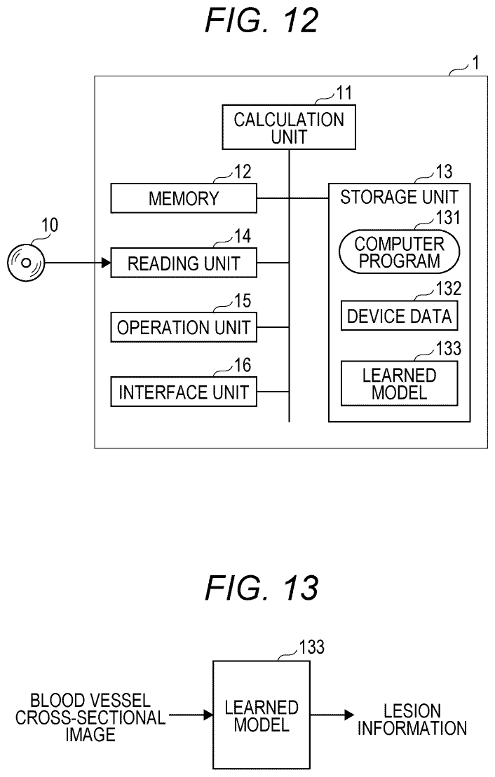

Note that the information processing apparatus 1 may execute processing using a learned model in S7. FIG. 12 is a block diagram illustrating an internal functional configuration example of the information processing apparatus 1 using the learned model. In this mode, the information processing apparatus 1 includes a learned model 133 used to acquire the lesion information. The learned model 133 is implemented by the calculation unit 11 executing information processing according to a computer program 131. The storage unit 13 stores data necessary for implementing the learned model 133.

The learned model 133 may be configured using hardware. For example, the learned model 133 may be configured using hardware including a processor and a memory that stores necessary programs and data. Alternatively, the learned model 133 may be implemented using a quantum computer. Alternatively, the learned model 133 may be provided outside the information processing apparatus 1, and the information processing apparatus 1 may execute processing using the external learned model 133. For example, the learned model 133 may be implemented using a cloud.

FIG. 13 is a conceptual diagram illustrating functions of the learned model 133. The learned model 133 is learned in advance so that lesion information including a type and a severity of the lesion portion is output in a case where the blood vessel cross-sectional image is input. For example, the learned model 133 is configured using a neural network such as a convolutional neural network (CNN), a long short-term memory (LSTM), or a transformer. The learned model 133 may be a model using a method other than the neural network.

The learned model 133 is generated by machine learning using training data. The learned model 133 is learned by a learning device using a computer. The training data includes a plurality of data sets in which the blood vessel cross-sectional image and the lesion information are associated with each other. For example, the lesion information included in the training data is information including a type and a severity of the lesion portion specified by executing predetermined image processing on the blood vessel cross-sectional image. For example, the lesion information included in the training data is information including a result of determining the type and the severity of the lesion portion based on the blood vessel cross-sectional image, by a person.

In machine learning, the learning device inputs the blood vessel cross-sectional image included in the training data to a model to be a base of the learned model 133, and the model performs calculation in response to the input of the blood vessel cross-sectional image and outputs the lesion information. The learning device adjusts a parameter of the calculation of the model, so as to reduce an error between the lesion information output by the model and a lesion result associated with the input blood vessel cross-sectional image. For example, the parameters are adjusted by a backpropagation method.

The learning device performs machine learning by repeating the processing using the plurality of data sets included in the training data and adjusting the parameters of the model. The learned model 133 is generated by adjusting the parameters of the calculation in this manner. For example, the adjusted final parameters are stored in the storage unit 13, and the calculation unit 11 executes information processing using the parameters, thereby the learned model 133 is implemented.

In this mode, the information processing apparatus 1 executes processing using the learned model 133, in the processing in S7. In S7, the calculation unit 11 inputs the generated blood vessel cross-sectional image to the learned model 133. The learned model 133 outputs the lesion information, in response to the input of the blood vessel cross-sectional image. The calculation unit 11 acquires the lesion information output by the learned model 133. In this mode, the lesion information is acquired, and the processing using the lesion information can be executed. By using the learned model 133, it is possible to easily acquire the lesion information.

The present disclosure is not limited to the content of the above-described embodiments, and various modifications can be made within the scope indicated in the claims. In other words, embodiments obtained by combining technical means appropriately changed within the scope indicated in the claims are also included in the technical scope of the present disclosure.

The matters described in each embodiment can be combined with each other. In addition, some or all of the independent claims and their dependent claims described in the claims can be combined with together, regardless of their dependent relationships. Furthermore, although a form (multiple dependent claim form) in which a claim dependent on two or more other claims is described is used in the claims, the claim form is not limited thereto. The present disclosure may be described using a form in which a multiple dependent claim dependent on at least one multiple dependent claim is described.

The detailed description above describes embodiments of a non-transitory computer-readable medium storing a computer program, an information processing apparatus, and an information processing method related to intravascular treatment. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A non-transitory computer-readable medium storing a computer program for causing a computer to execute processing comprising:
acquiring lesion information indicating a state of a lesion portion included in a blood vessel of a patient and bias information of a wire to be inserted into the blood vessel; and
specifying a recommended device recommended to be used for treatment on the lesion portion, based on the lesion information and the bias information, and device information of a treatment device to be used for intravascular treatment.

2. The non-transitory computer-readable medium according to claim 1, further comprising:
displaying a blood vessel cross-sectional image representing a cross section of the blood vessel; and
superimposing and displaying a direction and a strength of pressing the wire against an inner surface of the blood vessel at a position where the blood vessel cross-sectional image is obtained, included in the bias information, on the blood vessel cross-sectional image.

3. The non-transitory computer-readable medium according to claim 1, further comprising:
displaying a blood vessel cross-sectional image representing a cross section of the blood vessel;
displaying a fluoroscopic image in which the blood vessel is fluoroscopically viewed from outside of the patient; and
superimposing and displaying a trajectory of a plurality of positions in the blood vessel where the blood vessel cross-sectional image is created and a position where the displayed blood vessel cross-sectional image is created, on the fluoroscopic image.

4. The non-transitory computer-readable medium according to claim 3, further comprising:
superimposing and displaying a position of the lesion portion on the fluoroscopic image.

5. The non-transitory computer-readable medium according to claim 1, further comprising:
displaying a type and a severity of the lesion portion included in the lesion information.

6. The non-transitory computer-readable medium according to claim 1, further comprising:
displaying the specified recommended device.

7. The non-transitory computer-readable medium according to claim 1, further comprising:
displaying a blood vessel cross-sectional image representing a cross section of the blood vessel; and
superimposing and displaying a treatment range by the specified recommended device on the blood vessel cross-sectional image.

8. The non-transitory computer-readable medium according to claim 1, further comprising:
displaying a blood vessel cross-sectional image representing a cross section of the blood vessel;
receiving designation of a use device to be used for treatment on the lesion portion; and
superimposing and displaying a treatable range of the lesion portion by the use device on the blood vessel cross-sectional image.

9. The non-transitory computer-readable medium according to claim 8, further comprising:
displaying a fluoroscopic image in which the blood vessel is fluoroscopically viewed from outside of the patient; and
superimposing and displaying a position of the use device in the blood vessel when the use device is used, on the fluoroscopic image.

10. The non-transitory computer-readable medium according to claim 1, further comprising:
making a display indicating that a wire bias is inappropriate, in a case where the bias information is not suitable for the intravascular treatment.

11. The non-transitory computer-readable medium according to claim 10, further comprising:
displaying a proposal of processing for enabling the intravascular treatment, including a change in a route through which the wire passes in the blood vessel and a change in the wire.

12. The non-transitory computer-readable medium according to claim 1, further comprising:
acquiring the bias information, by generating the bias information including a direction and a strength of pressing the wire against an inner surface of the blood vessel at each position of the blood vessel, based on data representing a three-dimensional shape of the blood vessel created based on a fluoroscopic image, data representing a three-dimensional shape of the wire in a state of being inserted into the blood vessel created based on a fluoroscopic image in a state where the wire is inserted into the blood vessel, and information indicating elasticity of the wire.

13. The non-transitory computer-readable medium according to claim 1, further comprising:
acquiring a blood vessel cross-sectional image representing a cross section of the blood vessel; and
inputting the acquired blood vessel cross-sectional image to a learned model that outputs lesion information in a case where the blood vessel cross-sectional image is input and acquiring the lesion information output by the learned model.

14. The non-transitory computer-readable medium according to claim 1, further comprising:
specifying the recommended device, according to a type and a severity of the lesion portion included in the lesion information and a direction and a strength of pressing the wire against an inner surface of the blood vessel, included in the bias information.

15. An information processing apparatus comprising:
a processor configured to:
acquire lesion information indicating a state of a lesion portion included in a blood vessel of a patient and bias information of a wire to be inserted into the blood vessel; and
specify a recommended device recommended to be used for treatment on the lesion portion, based on the lesion information and the bias information, and device information of a treatment device to be used for intravascular treatment.

16. An information processing method comprising:
acquiring lesion information indicating a state of a lesion portion included in a blood vessel of a patient and bias information of a wire to be inserted into the blood vessel; and
specifying a recommended device recommended to be used for treatment on the lesion portion, based on the lesion information and the bias information, and device information of a treatment device to be used for intravascular treatment.

17. The method according to claim 16, further comprising:
displaying a blood vessel cross-sectional image representing a cross section of the blood vessel; and
superimposing and displaying a direction and a strength of pressing the wire against an inner surface of the blood vessel at a position where the blood vessel cross-sectional image is obtained, included in the bias information, on the blood vessel cross-sectional image.

18. The method according to claim 16, further comprising:
displaying a blood vessel cross-sectional image representing a cross section of the blood vessel;
displaying a fluoroscopic image in which the blood vessel is fluoroscopically viewed from outside of the patient; and
superimposing and displaying a trajectory of a plurality of positions in the blood vessel where the blood vessel cross-sectional image is created and a position where the displayed blood vessel cross-sectional image is created, on the fluoroscopic image.

19. The method according to claim 18, further comprising:
superimposing and displaying a position of the lesion portion on the fluoroscopic image.

20. The method according to claim 18, further comprising:
displaying a type and a severity of the lesion portion included in the lesion information.
